(19) United States
(12) Patent Application Publication
(10) Pub. No.: US 2015/0080732 A1
(43) Pub. Date: Mar. 19, 2015

(54) ULTRASOUND DIAGNOSTIC APPARATUS AND DATA PROCESSING METHOD

(71) Applicant: FUJIFILM CORPORATION, Tokyo (JP)

(72) Inventor: Hiroaki YAMAMOTO, Ashigara-kami-gun (JP)

(73) Assignee: FUJIFILM CORPORATION, Tokyo (JP)

(21) Appl. No.: 14/551,774

(22) Filed: Nov. 24, 2014

Related U.S. Application Data
(63) Continuation of application No. PCT/JP2013/064486, filed on May 24, 2013.

(30) Foreign Application Priority Data
May 25, 2012 (JP) 2012-120014

(51) Int. Cl. A61B 8/08; A61B 8/00; A61B 8/14

(52) U.S. Cl. CPC A61B 8/5269; A61B 8/14; A61B 8/4488; A61B 8/5207
USPC 600/447; 600/443

(57) ABSTRACT

In the ultrasound diagnostic apparatus, the ultrasonic wave transmitter/receiver transmits an ultrasonic beam to a subject, receives an ultrasonic echo that is a reflected ultrasonic beam from the subject, and outputs reception data; and the ambient sound speed calculator acquires first reception data for calculating an ambient sound speed as a sound speed in the subject, which is data of a lower frequency than second reception data for producing a brightness image of the subject, and analyses the acquired first reception data to calculate the ambient sound speed.

### CROSS-REFERENCE TO RELATED APPLICATIONS

This application is a Continuation of PCT International Application No. PCT/JP2013/064486 filed on May 24, 2013, which claims priority under 35 U.S.C. §119(a) to Japanese Application No. 2012-120014 filed on May 25, 2012. Each of the above applications is hereby expressly incorporated by reference, in its entirety, into the present application.

### BACKGROUND OF THE INVENTION

The present invention relates to an ultrasound diagnostic apparatus and a data processing method for creating and displaying an ultrasound image of a diagnostic region of a subject using an ultrasonic wave.

An ultrasound diagnostic apparatus using an ultrasound image has hitherto been put into practical use in the field of medicine. In general, the ultrasound diagnostic apparatus has an ultrasound probe in which a transducer array is installed, and an apparatus body connected to the ultrasound probe. In the ultrasound diagnostic apparatus, an ultrasonic beam is transmitted from the ultrasound probe toward a subject, an ultrasonic echo, which is a reflected ultrasonic beam from the subject, is received by the ultrasound probe to acquire a reception signal, and the acquired reception signal is electrically processed by the apparatus body to produce an ultrasound image.

In the conventional ultrasound diagnostic apparatus, the value of the sound speed of ultrasonic wave set for the entire apparatus is fixed to a certain value assuming that the sound speed of ultrasonic wave in the living body of the subject is constant.

However, since the sound speed changes depending on differences in tissues such as a fat layer and a muscular layer in the living body, the sound speed of ultrasonic wave in the subject (hereinafter, referred to as an ambient sound speed) is not uniform. In addition, since the thickness of the fat layer or the muscular layer is different between a fat subject and a thin subject, there are individual differences in the ambient sound speed for each subject.

As described above, in the conventional ultrasound diagnostic apparatus, the sound speed of ultrasonic wave set for the entire apparatus (hereinafter, referred to as a set sound speed) is fixed to a certain value. In this case, the more the ambient sound speed, which is the sound speed in the subject, deviates from the set sound speed, the more the arrival time of the reflected wave (ultrasonic echo) deviates from the delay time set for the ultrasonic wave transmission/reception circuit. For this reason, there has been a problem in that the focusing is degraded, and accordingly, the quality of the obtained ultrasound image is degraded.

Meanwhile, as shown in FIG. 12, the first embodiment of JP 2011-92686 A discloses a data analyzer 700 in which a region-of-interest setting unit 701 sets a region of interest on an ultrasound image, a transmission focus controller 702 sends an instruction of transmission focusing to a CPU 100 so that a transmission circuit performs transmission focusing on the region of interest, thereby setting the position of transmission focus as pseudo-point reflection, a set sound speed designation unit 703 sets a start sound speed and an end sound speed of a set sound speed, a focus index calculator 704 calculates a focus index by performing reception focusing on reception data of the region of interest for each set sound speed of a predetermined step sound speed amount, and an ambient sound speed determination unit 705 determines an ambient sound speed of the region of interest based on the focus index.

In addition, JP 2011-92686 A discloses that a signal processor 502 performs reception focus processing using the ambient sound speed calculated by the data analyzer 700.

### SUMMARY OF THE INVENTION

In general, in order to obtain the spatial resolution of a brightness image (an ultrasound image), the brightness image is created by transmitting and receiving ultrasonic waves having relatively high frequencies to and from a subject.

However, in ultrasonic waves reflected from the region of interest, a phenomenon such as an aberration due to variously different paths of the ultrasonic waves (hereinafter, simply referred to as aberration) occurs. In the case of a high-frequency ultrasonic wave, the wavefront is likely to collapse by the influence of the aberration.

When transmitting and receiving ultrasonic waves to and from countless scattering points in a speckle region as disclosed in JP 2011-92686 A, the wavefront collapses to a larger extent as the frequency of the ultrasonic wave becomes higher.

In the method disclosed in JP 2011-92686 A, since the collapse of the wavefront of the ultrasonic wave causes an error when calculating the ambient sound speed, it is not possible to calculate the ambient sound speed accurately. Therefore, there has been a problem in that, even if reception focus processing is performed on a brightness image, the focusing may not be accurately performed.

The present invention has been made to solve the above-described problems in the prior art, and it is an object of the present invention to provide an ultrasound diagnostic apparatus and a data processing method capable of performing the focusing of a brightness image accurately by calculating accurate ambient sound speed regardless of the state of a diagnostic region of a subject.

To attain the above object, the present invention provides an ultrasound diagnostic apparatus, comprising:

an ultrasonic wave transmitter/receiver configured to transmit an ultrasonic beam to a subject, receive an ultrasonic echo that is a reflected ultrasonic beam from the subject, and output reception data; and an ambient sound speed calculator configured to acquire first reception data for calculating an ambient sound speed as a sound speed in the subject, which is data of a lower frequency than second reception data for producing a brightness image of the subject, and analyse the acquired first reception data to calculate the ambient sound speed.

Also, the present invention provides a data processing method, comprising steps of:

transmitting an ultrasonic beam to a subject, receiving an ultrasonic echo that is a reflected ultrasonic beam from the subject, and outputting reception data, by an ultrasonic wave transmitter/receiver; and acquiring first reception data for calculating an ambient sound speed as a sound speed in the subject, which is data of a lower frequency than second reception data for producing a brightness image of the subject, and analysing the acquired first reception data to calculate the ambient sound speed, by an ambient sound speed calculator.

In the present invention, since an ambient sound speed is calculated by using the first reception data of a lower frequency than the second reception data for producing a brightness image, the ambient sound speed is less influenced by aberration. Therefore, it is possible to accurately calculate the ambient sound speed regardless of the state of the diagnostic region of the subject. In addition, since a brightness image is produced by correcting the delay time of the high-frequency second reception data for producing a brightness image using the ambient sound speed calculated from the low-frequency first reception data, it is possible to perform focusing of the brightness image accurately. Therefore, it is possible to produce a high-quality brightness image that is less influenced by the non-uniformity of sound speed.

### BRIEF DESCRIPTION OF THE DRAWINGS

FIG. 1 is a block diagram showing the configuration of an embodiment of an ultrasound diagnostic apparatus for carrying out a data processing method of the present invention.

FIG. 2 is a block diagram showing the configuration of an ambient sound speed calculator shown in FIG. 1.

FIG. 3 is a flowchart showing the flow of the process in the ambient sound speed calculator shown in FIG. 2.

FIG. 4 is a diagram showing a state of reception focus processing performed on the reception data from point reflection.

FIG. 5 is a diagram showing a state of countless scattering points in a speckle region.

FIG. 6 is a diagram showing a state where pseudo-point reflection is formed by performing transmission focusing on countless scattering points in a speckle region.

FIG. 7 is a graph showing the focus index of each set sound speed.

FIG. 8 is a flowchart showing the flow of the process in a live mode of the ultrasound diagnostic apparatus shown in FIG. 1.

FIG. 9 is a conceptual diagram of a first embodiment showing the flow of reception focus processing performed in the ultrasound diagnostic apparatus shown in FIG. 1.

FIG. 10 is a conceptual diagram of a second embodiment showing the flow of reception focus processing performed in the ultrasound diagnostic apparatus shown in FIG. 1.

FIG. 11 is a conceptual diagram showing a state where the delay time of reception data is corrected based on an ambient sound speed.

FIG. 12 is a block diagram showing the configuration of a data analyzer disclosed in JP 2011-92686 A.

### DETAILED DESCRIPTION OF THE INVENTION

Hereinafter, an ultrasound diagnostic apparatus and a data processing method of the present invention will be described in detail based on preferred embodiments shown in the accompanying drawings.

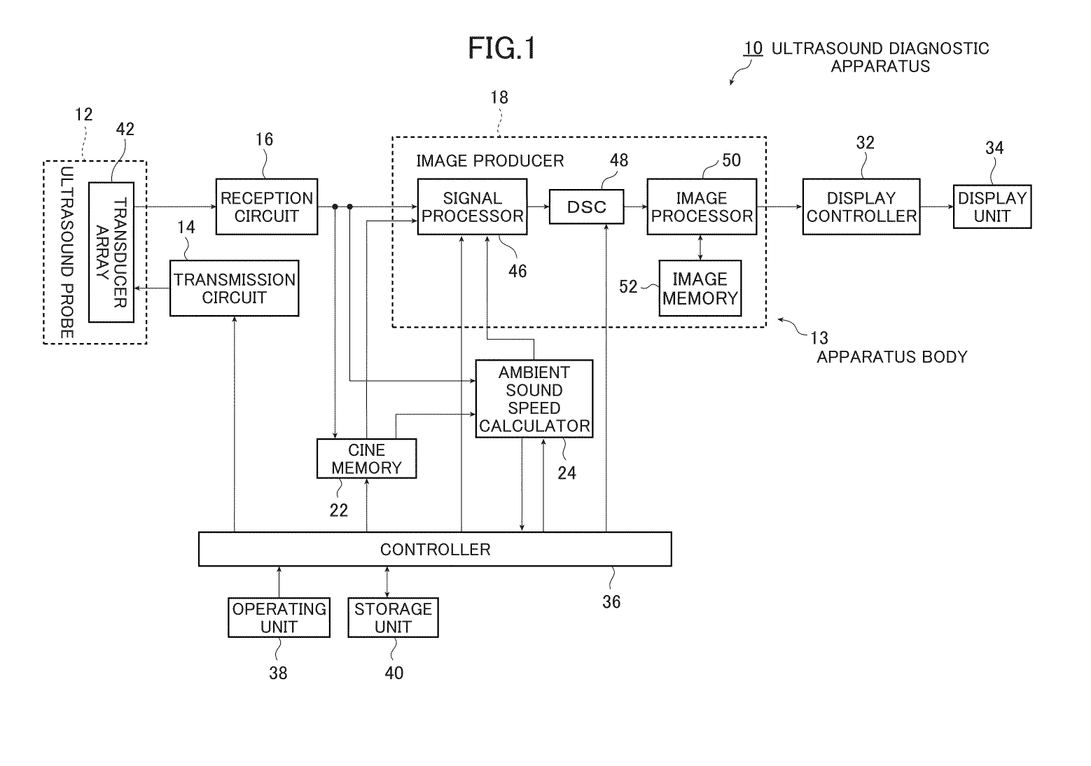

FIG. 1 is a block diagram showing the configuration of an embodiment of an ultrasound diagnostic apparatus for carrying out a data processing method of the present invention.

An ultrasound diagnostic apparatus 10 shown in FIG. 1 is configured with an ultrasound probe 12 and an apparatus body 13 connected to the ultrasound probe 12.

The apparatus body 13 includes a transmission circuit 14, a reception circuit 16, an image producer 18, a cine memory 22, an ambient sound speed calculator 24, a display controller 32, a display unit 34, a controller 36, an operating unit 38, and a storage unit 40.

The ultrasound diagnostic apparatus 10 is an apparatus which transmits an ultrasonic beam from the ultrasound probe 12 toward a subject, receives an ultrasonic echo which is a reflected ultrasonic beam from the subject, and produces and displays an ultrasound image from the reception signal of the ultrasonic echo.

The ultrasound probe 12 is used in a state of being brought into contact with a subject, and has a transducer array 42 which is used in a usual ultrasound diagnostic apparatus.

The transducer array 42 has a plurality of ultrasound transducers (ultrasonic wave transmission/reception elements) which are one-dimensionally or two-dimensionally arranged. When an ultrasound image is captured, each of the plurality of ultrasound transducers transmits an ultrasonic beam toward the subject in accordance with a driving signal supplied from the transmission circuit 14, receives an ultrasonic echo from the subject (that is, the ultrasonic beam reflected by the subject), and outputs a reception signal.

Each ultrasound transducer is constituted by a vibrator in which electrodes are formed at both ends of a piezoelectric substance formed of, for example, a piezoelectric ceramic represented by PZT (lead zirconate titanate), a polymer piezoelectric element represented by PVDF (polyvinylidene fluoride), a piezoelectric single crystal represented by PMN-PT (lead magnesium niobate-lead titanate solid solution), or the like.

If a pulsed or continuous-wave voltage is applied across the electrodes of the vibrator, the piezoelectric substance expands and contracts, whereby pulsed or continuous-wave ultrasonic waves are generated from the vibrator, and the generated ultrasonic waves are synthesized to form an ultrasonic beam. When receiving propagating ultrasonic wave, each vibrator expands and contracts to generate an electric signal and the electric signal is output as the reception signal of the ultrasonic wave.

Meanwhile, in the apparatus body 13, the transmission circuit 14 includes a plurality of pulsers, for example. The transmission circuit 14 performs transmission focusing to adjust the amount of delay of each driving signal (timing of applying a driving signal) so that ultrasonic waves transmitted from the plurality of ultrasound transducers of the transducer array 42 form an ultrasonic beam based on the transmission delay pattern selected by the controller 36, and supplies the adjusted driving signals to the plurality of ultrasound transducers. Thus, the ultrasonic beams are transmitted from the plurality of ultrasound transducers to the subject.

The reception circuit 16 amplifies the reception signal supplied from each ultrasound transducer of the transducer array 42, and A/D (analog/digital) converts the amplified reception signal to generate reception data (RF data).

The reception circuit 16 supplies the reception data to the image producer 18, the cine memory 22, and the ambient sound speed calculator 24.

The ultrasound probe 12, the transmission circuit 14, and the reception circuit 16 constitute an ultrasonic wave transmitter/receiver in the present invention.

Here, the transmission delay pattern is pattern data of the delay time that is given to a driving signal in order to form an ultrasonic beam in a desired direction with ultrasonic waves transmitted from the plurality of ultrasound transducers. The reception delay pattern is pattern data of the delay time that is given to a reception signal in order to extract an ultrasonic echo from a desired direction with ultrasonic waves received by the plurality of ultrasound transducers.

A plurality of transmission delay patterns and a plurality of reception delay patterns are stored in the storage unit 40 in advance. The controller 36 selects one transmission delay pattern and one reception delay pattern from the plurality of transmission delay patterns and the plurality of reception delay patterns stored in the storage unit 40 and outputs control signals to the transmission circuit 14 and a signal processor 46 according to the selected transmission delay pattern and reception delay pattern, thereby performing transmission/reception control of the ultrasonic wave.

Then, the image producer 18 produces an ultrasound image from the reception data supplied from the reception circuit 16.

As shown in FIG. 1, the image producer 18 includes the signal processor 46, a DSC 48, an image processor 50, and an image memory 52.

Since the distances between the respective ultrasound transducers and the ultrasonic reflection source in the subject are different, the time taken for the ultrasonic echo to reach each ultrasound transducer is different.

The signal processor 46 performs reception focus processing by correcting the difference in arrival time (delay time) of the ultrasonic echo in the reception data of a brightness image based on the ambient sound speed supplied from the ambient sound speed calculator 24 to be described later.

In the present embodiment, the signal processor 46 performs reception focus processing digitally by delaying each piece of reception data by the difference in arrival time (delay time) of the ultrasonic echo and performing phase matching addition on the delayed reception data.

When there is another ultrasonic reflection source at a position different from the position of the ultrasonic reflection source, the arrival time of the reception signal from the other ultrasonic reflection source is different. Therefore, the phase of the reception signal from the other ultrasonic reflection source is cancelled by addition in the signal processor 46. Thus, the reception signal from the ultrasonic reflection source becomes greatest, thereby becoming in focus. By the reception focus processing, the focus of the ultrasonic echo is narrowed down and reception data (sound ray signal) is generated.

Hereinafter, the correction of the delay time of the reception data by the signal processor 46 will be described.

FIG. 11 is a conceptual diagram showing a state where the delay time of reception data is corrected based on the ambient sound speed. As shown in FIG. 11, a case is considered in which a plurality of ultrasound transducers (ultrasonic wave transmission/reception elements) of the ultrasound probe 12 are arranged in a row in a horizontal direction in FIG. 11.

Here, assuming that the width of each ultrasound transducer in the arrangement direction of ultrasound transducers is L, the distance from the ultrasound transducer at the center in the arrangement direction to the n-th ultrasound transducer toward the end of the arrangement is nL.

As shown in FIG. 11, assuming that the reflection point of the ultrasonic wave is present at a position at a distance (depth) d from the ultrasound transducer at the center in a direction perpendicular to the arrangement direction, a distance (length) dₙ between the n-th ultrasound transducer and the reflection point is calculated by Expression (1).

dₙ=((nL)²+d²)^{1/2} (1)

Therefore, time tₙ until the ultrasonic wave from the reflection point is received by the n-th ultrasound transducer is calculated by Expression (2) using an ambient sound speed V.

tₙ=dₙ/V=((nL)²+d²)^{1/2}/V (2)

Since the distances between the respective ultrasound transducers and the reflection point are different as described above, in this example, as shown in the graph in the upper part of FIG. 11, the time tₙ becomes longer toward the ultrasound transducer at the end of the arrangement.

That is, assuming that the time until the ultrasonic wave from the reflection point is received by the ultrasound transducer at the center is t₁, the ultrasonic wave received by the n-th ultrasound transducer is delayed by time Δt=tₙ−t₁ with respect to the ultrasonic wave received by the ultrasound transducer at the center. The signal processor 46 corrects the delay time expressed by the time Δt for the reception data corresponding to each ultrasound transducer. This delay time Δt is referred to as a reception delay pattern. As described above, the delay time Δt of each piece of reception data is calculated from the ambient sound speed and the distance calculated from the geometric arrangement of the reflection point and each ultrasound transducer.

Although the example described above is a case where the ultrasound probe 12 is a linear probe, the same thinking can be applied to a convex probe except for the difference in the shape of the probe.

In addition, the signal processor 46 performs predetermined data processing on the reception data having been subjected to reception focus processing.

In the present embodiment, the signal processor 46 generates a B-mode image signal (image signal of a brightness image in which the amplitude of the ultrasonic echo is expressed by the brightness (luminance) of a point), which is tomographic image information regarding tissue within the subject, by performing correction of attenuation due to the distance depending on the depth of the reflection position of the ultrasonic wave and then performing envelope detection processing.

The B-mode image signal generated by the signal processor 46 is an image signal obtained by a scanning system different from a usual television signal scanning system.

Therefore, the DSC (a digital scan converter) 48 converts (raster-converts) the B-mode image signal generated by the signal processor 46 into a usual image signal, for example, an image signal according to the television signal scanning system (for example, an NTSC system) under the control of the controller 36.

The image processor 50 performs various necessary image processing, such as gradation processing, on the B-mode image signal input from the DSC 48, and then, the B-mode image signal after the image processing is stored in the image memory 52 and output to the display controller 32.

Subsequently, the display controller 32 causes the display unit 34 to display an ultrasound diagnostic image based on the B-mode image signal which has been subjected to image processing by the image processor 50.

The display unit 34 is, for example, a display device such as an LCD, and displays the ultrasound diagnostic image (a video and a still image) and various setting screens under the control of the display controller 32.

Then, the cine memory (reception data memory) 22 sequentially stores the reception data supplied from the reception circuit 16. In addition, the cine memory 22 stores information regarding the frame rate (for example, parameters indicating the depth of the reflection position of the ultrasonic wave, the density of scanning lines, and the width of a field of vision), which is input from the controller 36, so as to be associated with the reception data described above.

The reception data and the information regarding the frame rate stored in the cine memory 22 are supplied to the signal processor 46 and the ambient sound speed calculator 24.

The ambient sound speed calculator 24 acquires reception data for calculating the ambient sound speed, which is data of a lower frequency than reception data for producing a brightness image, and analyses the acquired reception data, thereby calculating an ambient sound speed.

In the present embodiment, the ambient sound speed calculator 24 acquires the reception data for calculating the ambient sound speed, calculates a focus index by performing reception focusing on the acquired reception data, and calculates the ambient sound speed in a region of interest based on the calculated focus index.

The ambient sound speed calculator 24 outputs the calculated ambient sound speed to the signal processor 46.

The controller 36 controls the respective constituents of the ultrasound diagnostic apparatus 10 on the basis of instructions input from the operating unit 38 by an operator.

The operating unit 38 is an input device for receiving instructions input by the operator, and may be constituted by a keyboard, a mouse, a trackball, a touch panel, or the like.

The storage unit 40 stores an operation program for causing the controller 36 to execute control of the respective constituents of the ultrasound diagnostic apparatus 10, the transmission delay pattern and reception delay pattern, or the like, and may be constituted by a recording medium such as a hard disk, a flexible disk, an MO, an MT, a RAM, a CD-ROM a DVD-ROM, or the like.

The signal processor 46, the DSC 48, the image processor 50, the display controller 32 and the ambient sound speed calculator 24 are constituted by a CPU (a computer) and an operation program for causing the CPU to execute various processing, but these may be constituted by digital circuits.

Next, the ambient sound speed calculator 24 will be described in detail.

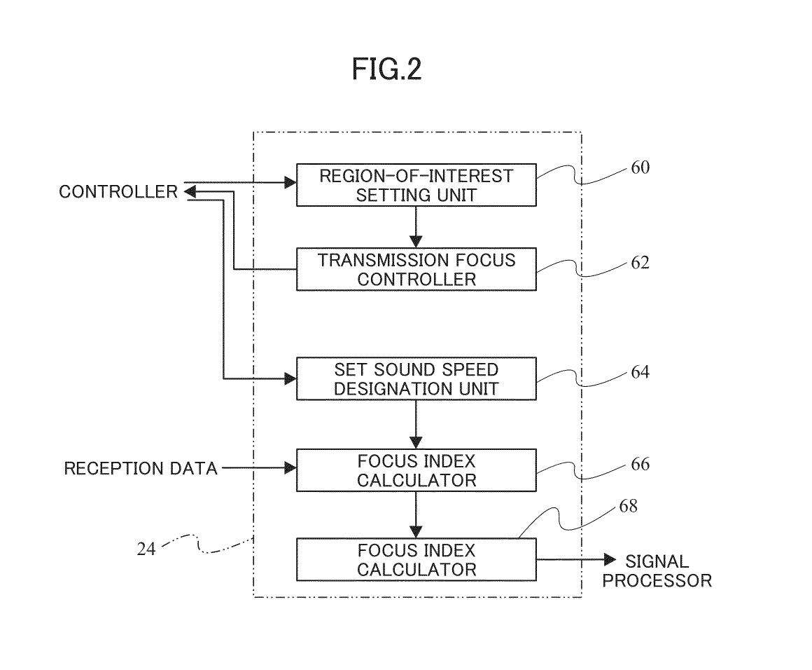

FIG. 2 is a block diagram showing the configuration of the ambient sound speed calculator shown in FIG. 1.

As shown in FIG. 2, the ambient sound speed calculator 24 includes a region-of-interest setting unit 60, a transmission focus controller 62, a set sound speed designation unit 64, a focus index calculator 66, and an ambient sound speed determination unit 68.

The region-of-interest setting unit 60 sets a region of interest on the ultrasound image displayed on the display unit 34 in response to an instruction input from the operating unit 38 and through the controller 36 by the operator.

The transmission focus controller 62 sends an instruction of transmission focusing to the controller 36 so that the transmission circuit 14 performs transmission focusing on the set region of interest.

The set sound speed designation unit 64 sequentially designates each of a plurality of set sound speeds for performing reception focusing on the reception data based on the control of the controller 36.

The focus index calculator 66 reads reception data of the region of interest from the cine memory 22, performs reception focusing on the reception data for each of a plurality of set sound speeds designated by the set sound speed designation unit 64, and calculates the focus indexes of the reception data.

The ambient sound speed determination unit 68 determines an ambient sound speed in the region of interest based on each of the focus indexes for each of the plurality of set sound speeds.

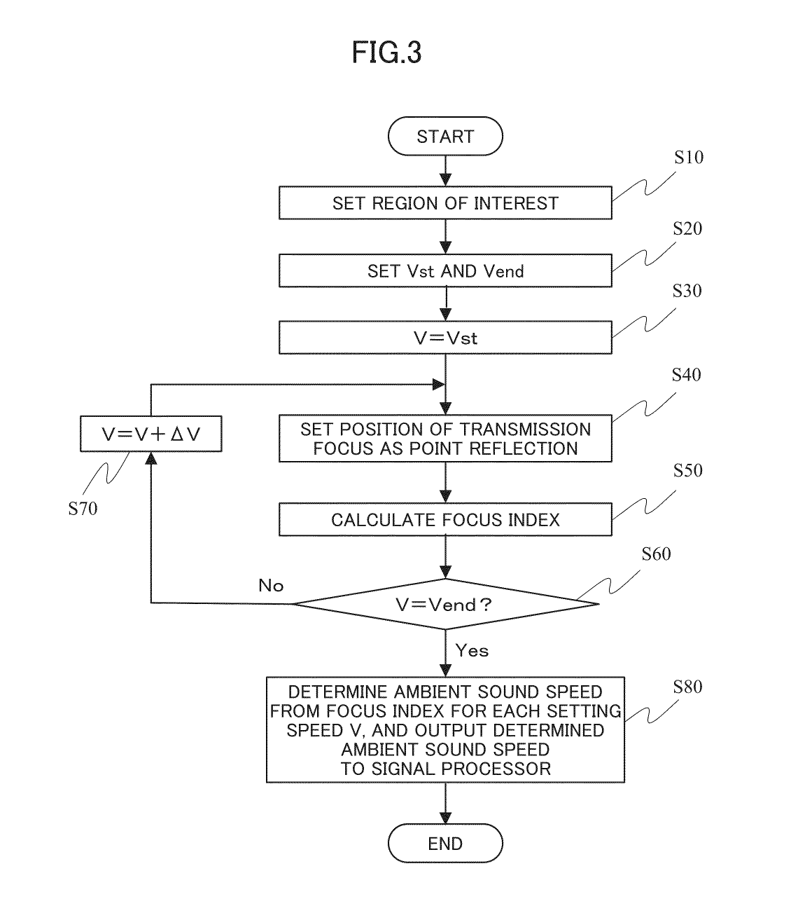

Next, the operation of the ambient sound speed calculator 24 will be described with reference to the flowchart of FIG. 3. FIG. 3 is a flowchart showing the flow of the process in the ambient sound speed calculator shown in FIG. 2.

As shown in FIG. 3, in the ambient sound speed calculator 24, the region-of-interest setting unit 60 sets a region of interest on the ultrasound image displayed on the display unit 34 in response to an instruction input by the operator using the operating unit 38 and through the controller 36 (step S10).

Then, in the ambient sound speed calculator 24, the set sound speed designation unit 64 sets a start sound speed Vst and an end sound speed Vend of the set sound speed V (step S20), and sets the start sound speed Vst as the set sound speed V (step S30).

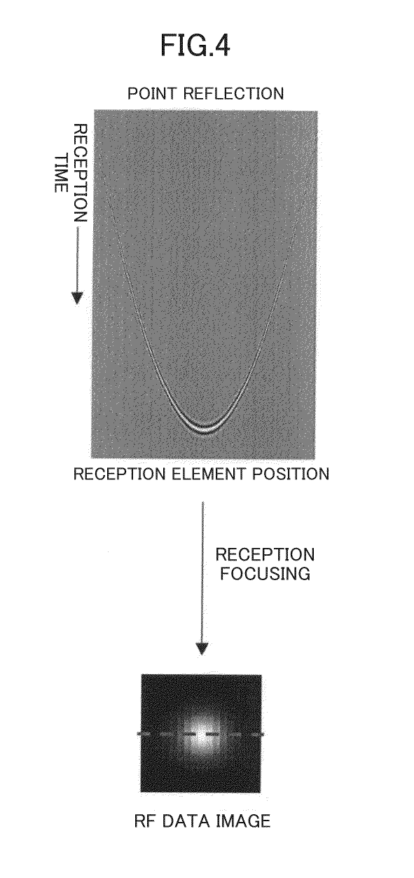

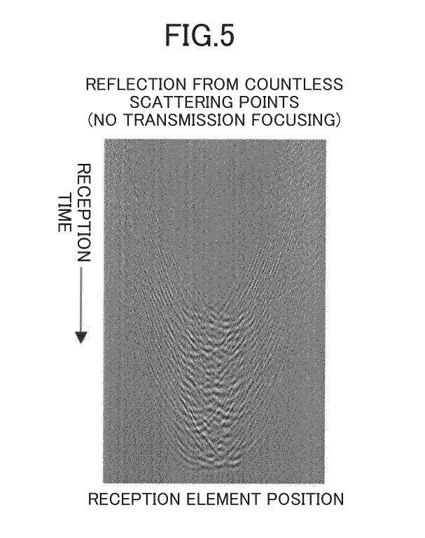

As shown in FIG. 4, with respect to reception data from point reflection, when performing reception focusing, it is possible to acquire reception data whose strength or sharpness can be analysed. However, as shown in FIG. 5, with respect to countless scattering points in a speckle region, the peak value and the spatial frequency in the azimuthal direction are no longer valid due to the interference. For this reason, it is difficult to acquire reception data whose strength or sharpness can be analysed when performing reception focusing.

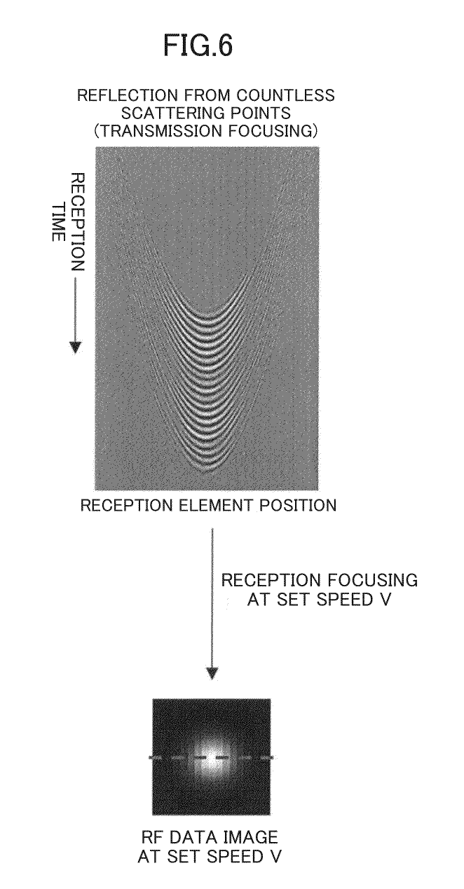

Therefore, as shown in FIG. 6, the ambient sound speed calculator 24 forms pseudo-point reflection by performing transmission focusing on the countless scattering points in a speckle region, performs reception focusing on the acquired reception data of the reception element position, and calculates an ambient sound speed in a speckle region using the same method as for the point reflection to analyse the strength or sharpness.

That is, in the ambient sound speed calculator 24, the transmission focus controller 62 sends an instruction of transmission focusing to the controller 36 so that the transmission circuit 14 performs transmission focusing on the set region of interest, thereby setting the position of transmission focusing as pseudo-point reflection (step S40).

Then, in the ambient sound speed calculator 24, the focus index calculator 66 reads the reception data from the cine memory 22, performs reception focusing on the reception data for each of the plurality of set sound speeds designated by the set sound speed designation unit 64, and calculates the focus index of the reception data (step S50).

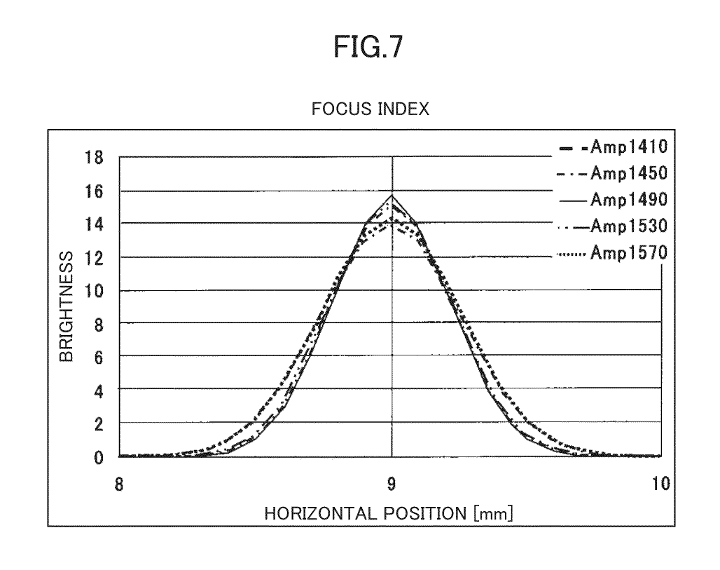

Here, in the case of the reception data of the point reflection in FIG. 4, as shown in FIG. 7, changes in the peak value and the spatial frequency in the azimuthal direction depending on the set sound speed can be seen. However, even in the case of reception data when forming pseudo-point reflection by performing transmission focusing as shown in FIG. 6, the tendency shown in FIG. 7 can be seen. For this reason, in the ambient sound speed calculator 24, the focus index calculator 66 calculates as focus indices an integration value, a square integration value, a peak value, contrast, half-value width, frequency spectrum integration, a frequency spectrum integration value or frequency spectrum square integration value standardized by a maximum value or a DC component, an autocorrelation value, and the like (in the case shown in FIG. 7, a focus index when the set sound speed=Amp1490 is the highest).

Then, in the ambient sound speed calculator 24, the set sound speed designation unit 64 determines whether or not the set sound speed V has reached the end sound speed Vend (step S60). If the set sound speed V is less than the end sound speed Vend (“No” in step S60), a predetermined step sound speed amount ΔV is added to the set sound speed V (step S70), and the process returns to step S40. On the other hand, if it is determined that the set sound speed V has reached the end sound speed Vend (“Yes” in step S60), the process proceeds to step S80.

In step S80, the ambient sound speed determination unit 68 of the ambient sound speed calculator 24 determines the ambient sound speed in the region of interest based on the focus index for each of the plurality of set sound speeds. For example, a set sound speed having the highest focus index is determined as the ambient sound speed in the region of interest. Then, the determined ambient sound speed is output to the signal processor 46, and the process ends (in the case of FIG. 7, the set sound speed=Amp1490 having the highest focus index is the ambient sound speed).

As described above, in the ultrasound diagnostic apparatus 10, transmission focusing is performed on the countless scattering points in a speckle region to form the pseudo-point reflection, a focus index for each of the plurality of set sound speeds is generated, and an ambient sound speed in the region of interest is determined based on the focus index for each of the plurality of set sound speeds. Therefore, it is possible to appropriately determine the ambient sound speed in the region of interest including a speckle region at the level of point reflection, and thus, it is possible to produce a highly accurate ultrasound image.

Next, the operation of the ultrasound diagnostic apparatus 10 will be described.

The ultrasound diagnostic apparatus 10 has two operation modes of a live mode and a cine memory reproduction mode.

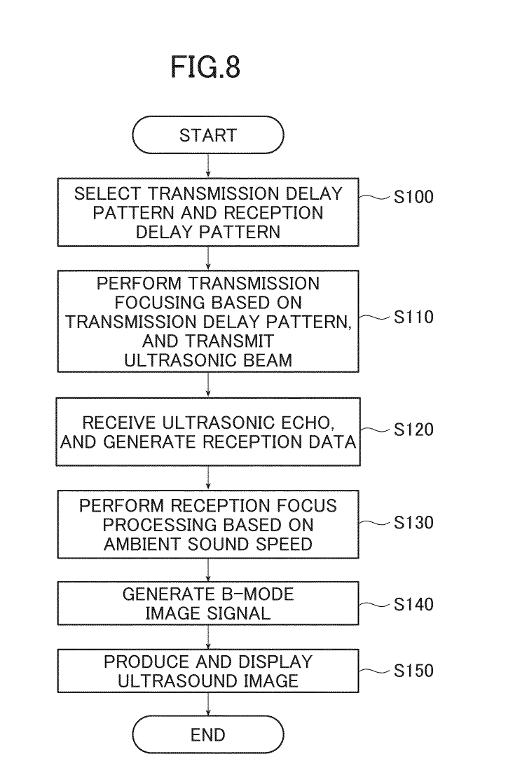

First, the operation of the ultrasound diagnostic apparatus 10 in the live mode will be described with reference to the flowchart shown in FIG. 8. FIG. 8 is a flowchart showing the flow of the process in the live mode of the ultrasound diagnostic apparatus shown in FIG. 1.

The live mode is a mode in which an ultrasound image (a video), which is obtained by transmitting and receiving ultrasonic waves in a state where the ultrasound probe 12 is brought into contact with the subject, is displayed.

In the live mode, the ultrasound probe 12 is brought into contact with the subject, and an ultrasound diagnosis is started by an instruction input from the operating unit 38 by the operator.

When the ultrasound diagnosis is started, the controller 36 sets a transmission direction of the ultrasonic beam and a reception direction of the ultrasonic echo for each ultrasound transducer, and selects a transmission delay pattern according to the transmission direction of the ultrasonic beam and selects a reception delay pattern according to the reception direction of the ultrasonic echo (step S100). Then, the controller 36 outputs control signals to the transmission circuit 14 and the signal processor 46 according to the selected transmission delay pattern and the selected reception delay pattern, thereby performing transmission/reception control of the ultrasonic wave.

In response to this, in the transmission circuit 14, transmission focusing of the driving signal of each ultrasound transducer is performed based on the selected transmission delay pattern, and the ultrasonic beams are transmitted from the plurality of ultrasound transducers to the subject (step S110).

Then, the ultrasonic echoes from the subject are received by the plurality of ultrasound transducers, and the reception signals are output from the plurality of ultrasound transducers.

In the reception circuit 16, the reception signal supplied from each ultrasound transducer is amplified and A/D converted to generate reception data (step S120).

Subsequently, in the signal processor 46, reception focus processing is digitally performed on the reception data based on the ambient sound speed supplied from the ambient sound speed calculator 24, thereby generating reception data in which the focus of the ultrasonic echo is narrowed down (step S130). Then, by the signal processor 46, data processing is performed on the reception data which has been subjected to the reception focus processing to generate a B-mode image signal (step S140).

In the DSC 48, the B-mode image signal generated by the signal processor 46 is converted into an image signal according to the television signal scanning system, and then, in the image processor 50, the converted B-mode image signal is subjected to necessary image processing to produce an ultrasound image.

The ultrasound image produced by the image processor 50 is stored in the image memory 52, and is displayed on the display unit 34 under the control of the display controller 32 (step S150).

In addition, the reception data generated by the reception circuit 16 is sequentially stored in the cine memory 22 so as to be associated with the information regarding the frame rate input from the controller 36, and is supplied to the ambient sound speed calculator 24 in order to generate the ambient sound speed.

As described above, in the ultrasound diagnostic apparatus 10, the ambient sound speed calculator 24 calculates the ambient sound speed in the region of interest of the subject, and reception focus processing is performed using the calculated ambient sound speed. In this case, the ambient sound speed is calculated from reception data of an ultrasound image having a lower frequency than reception data of a brightness image, and reception focus processing is performed on the reception data for producing a brightness image having a higher frequency than the reception data of an ultrasound image for calculating an ambient sound speed.

That is, the reception data for calculating an ambient sound speed is data of a lower frequency than the reception data for producing a brightness image.

Here, the method of acquiring a low-frequency ultrasound image for calculating an ambient sound speed is not particularly limited. Two examples for acquiring a low-frequency ultrasound image will be described below.

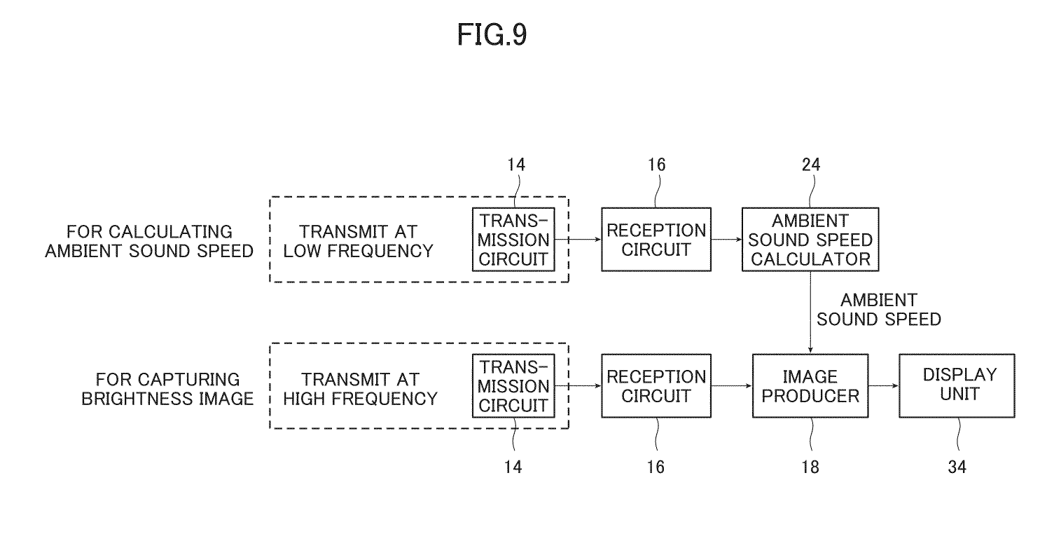

FIG. 9 is a conceptual diagram of the first embodiment showing the flow of reception focus processing performed in the ultrasound diagnostic apparatus shown in FIG. 1.

In the first embodiment, an ambient sound speed in the region of interest of the subject is calculated in advance, and when producing a brightness image, reception focus processing is performed on the reception data of a brightness image using the calculated ambient sound speed.

As shown in FIG. 9, when acquiring reception data for calculating an ambient sound speed, under the control of the controller 36, an ultrasonic beam having a lower frequency than an ultrasonic beam that is transmitted to the subject in order to acquire the reception data of a brightness image is transmitted to the subject by the transmission circuit 14, and an ultrasonic echo from the subject is received by the reception circuit 16. As a result, the reception data for calculating an ambient sound speed is acquired. Then, the ambient sound speed calculator 24 analyses the acquired reception data and calculates an ambient sound speed.

For example, in the case of acquiring the reception data of the abdominal region, the frequency of an ultrasonic beam when acquiring the reception data for calculating an ambient sound speed is preferably 2 MHz or lower.

In contrast, when acquiring reception data of a brightness image, under the control of the controller 36, an ultrasonic beam having a higher frequency than that when acquiring the reception data for calculating an ambient sound speed is transmitted to the subject by the transmission circuit 14, and an ultrasonic echo from the subject is received by the reception circuit 16, thereby acquiring the reception data. Then, the image producer 18 performs reception focus processing on the acquired reception data of a brightness image using the calculated ambient sound speed, and a brightness image is displayed on the display unit 34.

In the first embodiment described above, the calculation of the ambient sound speed and the acquisition of the reception data of a brightness image are performed so as to correspond in a one-to-one manner. However, the present invention is not limited thereto, and the number of times by which the ambient sound speed calculator 24 calculates the ambient sound speed by acquiring the reception data for calculating an ambient sound speed may be reduced to less than the number of times by which the reception data of a brightness image is acquired. For example, it is preferable to calculate the ambient sound speed when a diagnostic region is changed. When the diagnostic region remains the same, the reception focus processing may be performed on the acquired reception data of a brightness image using the same ambient sound speed (the ambient sound speed calculated in the diagnostic region).

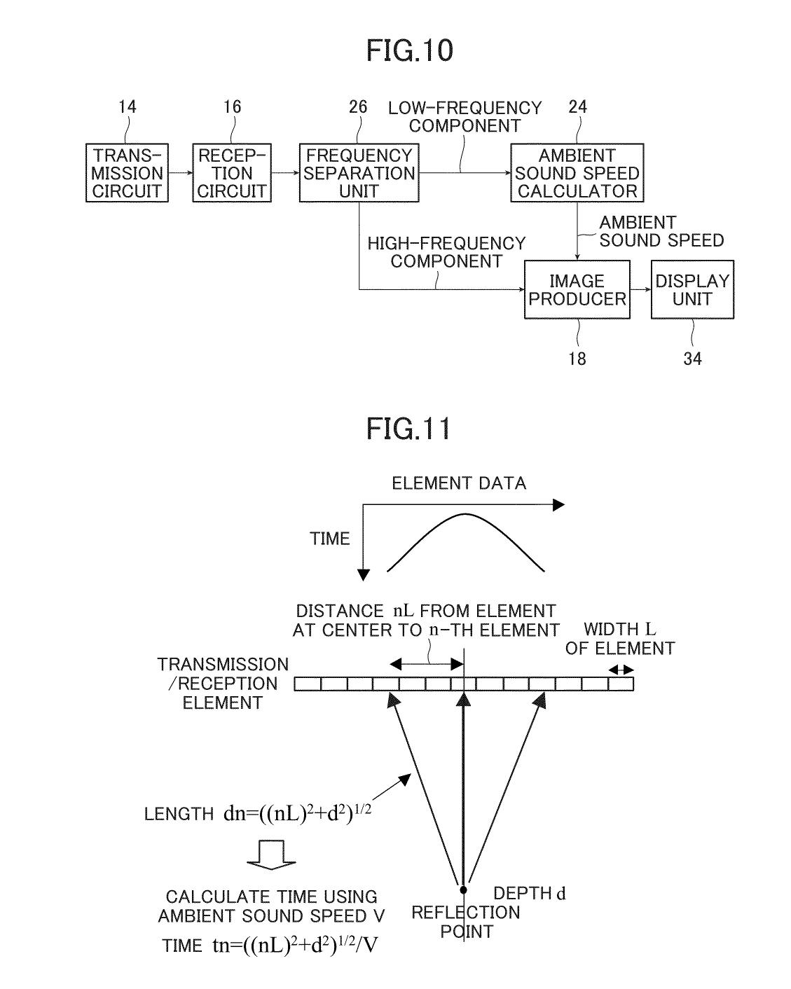

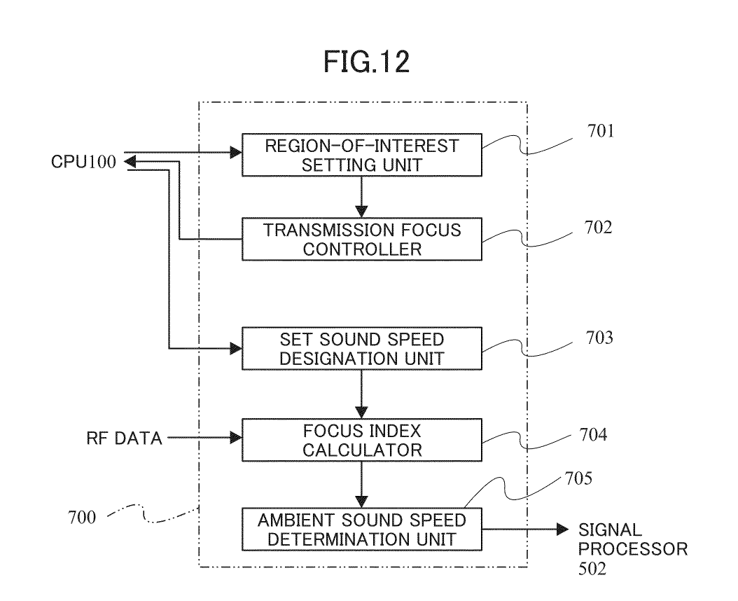

Next, FIG. 10 is a conceptual diagram of a second embodiment showing the flow of reception focus processing performed in the ultrasound diagnostic apparatus shown in FIG. 1.

In the second embodiment, frequency separation is performed on the reception data of a brightness image in the first embodiment, thereby extracting a low-frequency component and a high-frequency component. Then, an ambient sound speed is calculated from the low-frequency component, and reception focus processing is performed on the high-frequency component using the calculated ambient sound speed. As a result, a brightness image is produced.

As shown in FIG. 10, under the control of the controller 36, the transmission circuit 14 and the reception circuit 16 transmit and receive an ultrasonic beam to and from the subject, thereby acquiring the reception data. Then, from the acquired reception data, a frequency separation unit 26 extracts a component (low-frequency component) corresponding to reception data for calculating an ambient sound speed, which has a lower frequency than the ultrasonic beam transmitted to the subject in order to acquire the reception data, and other high-frequency components corresponding to reception data for producing a brightness image. For the separation of frequency components, for example, a method of using a band pass filter simply can be considered.

Similarly to the above, in the case of acquiring the reception data of the abdominal region, the frequency of the low-frequency component corresponding to the reception data of an ultrasound image for calculating an ambient sound speed is preferably 2 MHz or lower.

Subsequently, the ambient sound speed calculator 24 calculates an ambient sound speed from the extracted low-frequency component. On the other hand, the image producer 18 performs reception focus processing on the reception data of the extracted high-frequency component using the calculated ambient sound speed, and the high-frequency component after the reception focus processing, that is, a brightness image is displayed on the display unit 34.

In the second embodiment, although the high-frequency component extracted from the acquired reception data is used as reception data for producing a brightness image, the acquired reception data may be used as it is instead of the high-frequency component extracted from the reception data.

As described above, since an ambient sound speed is calculated by using the reception data of a lower frequency than the reception data for producing a brightness image, the ambient sound speed is less influenced by aberration. Therefore, it is possible to accurately calculate the ambient sound speed regardless of the state of the diagnostic region of the subject. In addition, since a brightness image is produced by correcting the delay time of the high-frequency reception data for producing a brightness image using the ambient sound speed calculated from the low-frequency reception data, it is possible to perform reception focusing accurately. Therefore, it is possible to produce a high-quality brightness image that is less influenced by the non-uniformity of sound speed.

Next, the operation of the ultrasound diagnostic apparatus 10 in the cine memory reproduction mode will be described.

The cine memory reproduction mode is a mode in which an ultrasound image is displayed based on the reception data stored in the cine memory 22.

In response to an instruction input from the operating unit 38, the controller 36 switches the operation mode of the ultrasound diagnostic apparatus 10 to the cine memory reproduction mode.

In the cine memory reproduction mode, the controller 36 reads reception data from the cine memory 22, and transmits the reception data to the signal processor 46 of the image producer 18. The subsequent operation is the same as in the live mode. Thus, an ultrasound image (a video or a still image) based on the reception data stored in the cine memory 22 is displayed on the display unit 34.

In addition, when the ambient sound speed calculator 24 calculates an ambient sound speed from the reception data, it is possible to use any method of calculating an ambient sound speed without being limited to the embodiments described above.

The present invention is basically as described above.

Hereinbefore, the present invention has been described in detail, but needless to say, the present invention is not limited to the above-described embodiments, and may be improved or modified in various ways within a scope that does not depart from the gist of the present invention.

What is claimed is:

1. An ultrasound diagnostic apparatus, comprising:
an ultrasonic wave transmitter/receiver configured to transmit an ultrasonic beam to a subject, receive an ultrasonic echo that is a reflected ultrasonic beam from the subject, and output reception data; and
an ambient sound speed calculator configured to acquire first reception data for calculating an ambient sound speed as a sound speed in the subject, which is data of a lower frequency than second reception data for producing a brightness image of the subject, and analyse the acquired first reception data to calculate the ambient sound speed.

2. The ultrasound diagnostic apparatus according to claim 1,
wherein the first reception data is acquired by transmitting an ultrasonic beam, which has a lower frequency than an ultrasonic beam transmitted to the subject in order to acquire the second reception data, to the subject.

3. The ultrasound diagnostic apparatus according to claim 1,
wherein the first reception data is acquired by extracting a low-frequency component from the second reception data.

4. The ultrasound diagnostic apparatus according to claim 2,
wherein a low frequency when acquiring the first reception data is a frequency of 2 MHz or lower.

5. The ultrasound diagnostic apparatus according to claim 1,
wherein a number of times by which the ambient sound speed calculator acquires the first reception data to calculate the ambient sound speed is equal to or less than a number of times by which the second reception data is acquired.

6. The ultrasound diagnostic apparatus according to claim 1,
wherein the ambient sound speed calculator comprises:
a region-of-interest setting unit configured to set a region of interest on an ultrasound image in response to an instruction input from an operating unit by an operator;
a transmission focus controller configured to control so as to perform transmission focusing on the region of interest to adjust an amount of delay of driving signals of a plurality of ultrasonic wave transmission/reception elements included in the ultrasonic wave transmitter/receiver;
a set sound speed designation unit configured to sequentially designate each of a plurality of set sound speeds for performing reception focusing on the first reception data to correct a difference in arrival time of the ultrasonic echo in the first reception data based on the ambient sound speed;
a focus index calculator configured to perform the reception focusing on the first reception data of the region of interest for each of the plurality of set sound speeds, and calculate focus indexes of the first reception data; and
an ambient sound speed determination unit configured to determine an ambient sound speed in the region of interest based on each of the focus indexes for each of the plurality of set sound speeds.

7. The ultrasound diagnostic apparatus according to claim 6,
wherein the ambient sound speed determination unit determines a set sound speed having a highest focus index as the ambient sound speed in the region of interest.

8. The ultrasound diagnostic apparatus according to claim 1, further comprising a signal processor configured to produce the brightness image by correcting a difference in arrival time of the ultrasonic echo in the second reception data based on the ambient sound speed and performing data processing on the corrected second reception data.

9. The ultrasound diagnostic apparatus according to claim 8,
wherein the signal processor corrects the difference in arrival time of the ultrasonic echo in the second reception data by delaying the second reception data corresponding to each of a plurality of ultrasonic wave transmission/reception elements included in the ultrasonic wave transmitter/receiver by the difference in arrival time of the ultrasonic echo in the second reception data, and performing phase matching addition on the delayed second reception data corresponding to each of the plurality of ultrasonic wave transmission/reception elements.

10. The ultrasound diagnostic apparatus according to claim 9,
wherein the signal processor calculates time until the ultrasonic echo from a reflection point is received by each of the plurality of ultrasonic wave transmission/reception elements based on the ambient sound speed and distances between the respective ultrasonic wave transmission/reception elements and the reflection point of the ultrasonic echo which is present at a position perpendicular to an arrangement direction of the plurality of ultrasonic wave transmission/reception elements from a reference ultrasonic wave transmission/reception element of the plurality of ultrasonic wave transmission/reception elements, and calculates the difference in arrival time of the ultrasound echo corresponding to each of the plurality of ultrasonic wave transmission/reception elements in the second reception data by calculating the difference between time until the ultrasonic echo from the reflection point is received by the reference ultrasonic wave transmission/reception element and the time until the ultrasonic echo from the reflection point is received by each of the plurality of ultrasonic wave transmission/reception elements.

11. A data processing method, comprising steps of:
transmitting an ultrasonic beam to a subject, receiving an ultrasonic echo that is a reflected ultrasonic beam from the subject, and outputting reception data, by an ultrasonic wave transmitter/receiver; and
acquiring first reception data for calculating an ambient sound speed as a sound speed in the subject, which is data of a lower frequency than second reception data for producing a brightness image of the subject, and analysing the acquired first reception data to calculate the ambient sound speed, by an ambient sound speed calculator.

12. The data processing method according to claim 11,
wherein the first reception data is acquired by transmitting an ultrasonic beam, which has a lower frequency than an ultrasonic beam transmitted to the subject in order to acquire the second reception data, to the subject.

13. The data processing method according to claim 11,
wherein the first reception data is acquired by extracting a low-frequency component from the second reception data.

14. The data processing method according to claim 12,
wherein a low frequency when acquiring the first reception data is a frequency of 2 MHz or lower.

15. The data processing method according to claim 11,
wherein a number of times by which the ambient sound speed calculator acquires the first reception data to calculate the ambient sound speed is equal to or less than a number of times by which the second reception data is acquired.

16. The data processing method according to claim 11,
wherein the step of calculating the ambient sound speed by the ambient sound speed calculator comprises the steps of:
setting a region of interest on an ultrasound image in response to an instruction input from an operating unit by an operator, by a region-of-interest setting unit;
controlling so as to perform transmission focusing on the region of interest to adjust an amount of delay of driving signals of a plurality of ultrasonic wave transmission/reception elements included in the ultrasonic wave transmitter/receiver, by a transmission focus controller;
sequentially designating each of a plurality of set sound speeds for performing reception focusing on the first reception data to correct a difference in arrival time of the ultrasonic echo in the first reception data based on the ambient sound speed, by a set sound speed designation unit;
performing the reception focusing on the first reception data of the region of interest for each of the plurality of set sound speeds, and calculating focus indexes of the first reception data, by a focus index calculator; and
determining an ambient sound speed in the region of interest based on each of the focus indexes for each of the plurality of set sound speeds, by an ambient sound speed determination unit.

17. The data processing method according to claim 16,
wherein the ambient sound speed determination unit determines a set sound speed having a highest focus index as the ambient sound speed in the region of interest.

18. The data processing method according to claim 11, further comprising a step of producing the brightness image by correcting a difference in arrival time of the ultrasonic echo in the second reception data based on the ambient sound speed and performing data processing on the corrected second reception data, by a signal processor.

19. The data processing method according to claim 18,
wherein the signal processor corrects the difference in arrival time of the ultrasonic echo in the second reception data by delaying the second reception data corresponding to each of a plurality of ultrasonic wave transmission/reception elements included in the ultrasonic wave transmitter/receiver by the difference in arrival time of the ultrasonic echo in the second reception data, and performing phase matching addition on the delayed second reception data corresponding to each of the plurality of ultrasonic wave transmission/reception elements.

20. The data processing method according to claim 19,
wherein the signal processor calculates time until the ultrasonic echo from a reflection point is received by each of the plurality of ultrasonic wave transmission/reception elements based on the ambient sound speed and distances between the respective ultrasonic wave transmission/reception elements and the reflection point of the ultrasonic echo which is present at a position perpendicular to an arrangement direction of the plurality of ultrasonic wave transmission/reception elements from a reference ultrasonic wave transmission/reception element of the plurality of ultrasonic wave transmission/reception elements, and calculates the difference in arrival time of the ultrasound echo corresponding to each of the plurality of ultrasonic wave transmission/reception elements in the second reception data by calculating the difference between time until the ultrasonic echo from the reflection point is received by the reference ultrasonic wave transmission/reception element and the time until the ultrasonic echo from the reflection point is received by each of the plurality of ultrasonic wave transmission/reception elements.
